(19) United States
(12) Patent Application Publication
(10) Pub. No.: US 2010/0130976 A1
(43) Pub. Date: May 27, 2010

(54) REDUCING CROSS-TALK EFFECTS IN AN RF ELECTROSURGICAL DEVICE

(75) Inventors: Ilya Bystryak, Salem, MA (US); Stanislav Polipas, Waltham, MA (US)

Correspondence Address: FISH & RICHARDSON P.C.;SMITH & NEPHEW, INC., 150 Minuteman Road, Andover, MA, 01810, US

(73) Assignee: Smith & Nephew Inc., Memphis, TN (US)

(21) Appl. No.: 12/622,102

(22) Filed: Nov. 19, 2009

Related U.S. Application Data
(60) Provisional application No. 61/116,933, filed on Nov. 21, 2008.

(51) Int. Cl. A61B 18/16

(52) U.S. Cl. 606/42

(57) ABSTRACT

A first probe and a second probe are coupled to a source of electrical energy. The first probe and the second probe are each configured to create a lesion when inserted into tissue and electrical energy is applied from the source of electrical energy. A first switch is coupled to the first probe and couples the first probe to ground when in a closed state. A second switch is coupled to the second probe and couples the second probe to ground when in a closed state. A control system is configured to receive an indication of a first parameter at the first probe and control the first switch based on the first parameter. The control system is also configured to receive an indication of a second parameter at the second probe and control the second switch based on the second parameter.

### CROSS-REFERENCE TO RELATED APPLICATIONS

This application claims priority to U.S. Provisional Application Ser. No. 61/116,933, filed on Nov. 21, 2008.

### TECHNICAL FIELD

This description is related to reducing the effects of cross-talk in a radiofrequency (RF) electrosurgical device.

### BACKGROUND

Radiofrequency (RF) ablation or lesioning is a technique that uses RF energy to produce heat to destroy tissue. This technique is used in a number of procedures, such as the lesioning of heart tissue to correct abnormal heartbeats and the destruction of tumors. RF lesioning is also used in procedure known as rhizotomy to treat pain, such as back pain, by stunning or destroying problematic spinal nerves. This procedure may be performed, for example, to treat pain caused by a herniated disc or from facet joint syndrome. The RF energy is transmitted through a probe placed adjacent to a sensory nerve. The RF energy produces heat to destroy the sensory nerve(s) carrying the pain.

### SUMMARY

In one aspect, an electrosurgical system includes a source of electrical energy, a grounding pad, a first probe, a second probe, a first switch, a second switch, and a control system. The grounding pad is coupled to the source of electrical energy and configured to be coupled to a body of a patient. The first probe is coupled to the source of electrical energy and configured to be inserted into tissue of the patient. In addition, the first probe is configured to create a lesion when the first probe is inserted into tissue and electrical energy is applied to the first probe from the source of electrical energy. Similarly, t second probe is coupled to the source of electrical energy and configured to be inserted into tissue of the patient. Also, the second dprobe is configured to create a lesion when the second probe is inserted into tissue and electrical energy is applied to the second probe from the source of electrical energy. The first switch is coupled to the first probe such that the first switch couples the first probe to ground when in a closed state and the second switch is coupled to the second probe such that the second switch couples the second probe to ground when in a closed state. The control system is configured to apply electrical energy from the source of electrical energy to the first probe in a manner that causes the first probe to create a lesion when the first probe is inserted into tissue and to apply electrical energy from the source of electrical energy to the second probe in a manner that causes the second probe to create a lesion when the second probe is inserted into tissue. Furthermore, the control system is configured to receive an indication of a first parameter associated with the first probe, control the first switch based on the first parameter, receive an indication of a second parameter associated with the second probe, and control the second switch based on the second parameter.

Implementations of any aspect may include one or more of the following features. For example, the first parameter may include a first temperature at the first probe such that the control system is configured to control the first switch based on the first temperature and the second parameter may include a second temperature at the second probe such that the control system is configured to control the second switch based on the second temperature. To control the first switch based on the first temperature, the control system may be configured to close the first switch when the first temperature is above a first value. To control the second switch based on the second temperature, the control system may be configured to close the second switch when the second temperature is above the first value. The control system may be configured to open the first switch when the first temperature is below the first value and to open the second switch when the second temperature is below the first value.

To apply electrical energy from the source of electrical energy to the first probe in a manner that causes the second probe to create a lesion when the first probe is inserted into tissue, the control system may be configured to apply electrical energy to the first probe when the first temperature is below a second value and remove the applied electrical energy from the first probe when the first temperature is above the second value. Tto apply electrical energy from the source of electrical energy to the second probe in a manner that causes the second probe to create a lesion when the second probe is inserted into tissue, the control system may be configured to apply electrical energy to the second probe when the second temperature is below the second value and remove the applied electrical energy from the second probe when the second temperature is above the second value.

The system may include a third switch and a fourth switch. The third switch may be coupled between the first probe and the source of electrical energy such that the first probe is disconnected from the source of electrical energy when the third switch is in an open state and connected to the source of electrical energy when the third switch is in a closed state The fourth switch may be coupled between the second probe and the source of electrical energy such that the second probe is disconnected from the source of electrical energy when the fourth switch is in an open state and connected to the source of electrical energy when the fourth switch is in a closed state. To apply electrical energy to the first probe, the control system may be configured to close the third switch and, to remove the applied electrical energy from the first probe, the control system is configured to open the third switch. To apply electrical energy to the second probe, the control system is configured to close the fourth switch and, to remove the applied electrical energy from the second probe, the control system is configured to open the fourth switch.

The control system may be configured to pulse width modulate the electrical energy applied to the first probe by opening and closing the third switch; and to pulse width modulate the electrical energy applied to the second probe by opening and closing the fourth switch.

To apply electrical energy to the first probe, the control system may be configured to cause the source of electrical energy to output a voltage with a non-zero magnitude and, to remove the applied electrical energy from the first probe, the control system may be configured to cause the source of electrical energy to output a voltage with a zero magnitude. To apply electrical energy to the second probe, the control system may be configured to cause the source of electrical energy to output a voltage with a non-zero magnitude and, to remove the applied electrical energy from the second probe, the control system may be configured to cause the source of electrical energy to output a voltage with a zero magnitude.

The first parameter may include a first current through the first probe such that the control system is configured to control the first switch based on the first current and the second parameter may include a second current through the second probe such that the control system is configured to control the second switch based on the second current. To control the first switch based on the first current, the control system may be configured to open the first switch when the first current is below a first value and close the first switch when the first current is above the first value. To control the second switch based on the second current, the control system may be configured to open the second switch when the second current is below the first value and close the second switch when the second current is above the first value.

The control system may be configured to close the third switch when the first parameter is below a first value, open the third switch when the first parameter is above the first value, close the fourth switch when the second parameter is below the first value, and open the fourth switch when the second parameter is above the first value. The control system may be configured to control an amount of power applied to the first probe or the second probe by controlling a magnitude of a voltage output by the source of electrical energy.

The first probe may include a first probe tip and the second probe may include a second probe tip. The first probe and first switch may be configured such that current flows from the first probe to ground without passing through the first probe tip when the first switch is closed. The second probe and second switch may be configured such that current flows from the second probe to ground without passing through the second probe tip when the first switch is closed.

The first probe and first switch may be configured such that an impedance between the first probe and ground is less than an impedance between the first probe and the grounding pad when the first probe is inserted in the tissue of the patient and the first switch is closed. The second probe and second switch may be configured such that an impedance between the second probe and ground is less than an impedance between the second probe and the grounding pad when the second probe is inserted in the tissue of the patient and the second switch is closed.

In another aspect, a method of performing electrosurgery may include coupling a grounding pad to a body of a patient, where the grounding pad is also coupled to a source of electrical energy. The method includes inserting a first probe into tissue of the patient and a second probe into tissue of the patient. The first probe and second probes are each coupled to the source of electrical energy and configured to create a lesion when inserted into tissue and electrical energy is applied from the source of electrical energy. The method further includes applying electrical energy from the source of electrical energy to the first probe in a manner that causes the first probe to create a lesion in the tissue into which the first probe is inserted and applying electrical energy from the source of electrical energy to the second probe in a manner that causes the second probe to create a lesion in the tissue into which the second probe is inserted. The method further includes receiving an indication of a first parameter associated with the first probe; controlling a first switch based on the first parameter, wherein the first switch is coupled to the first probe such that the first switch couples the first probe to ground when in a closed state; receiving an indication of a second parameter associated with the second probe; and controlling a second switch based on the second parameter, wherein the second switch is coupled to the second probe such that the second switch couples the second probe to ground when in a closed state;

Implementations of any aspect may include one or more of the following features. For example, the first parameter may include a first temperature at the first probe such that controlling the first switch comprises controlling the first switch based on the first temperature and the second parameter may include a second temperature at the second probe such that controlling the second switch comprises controlling the second switch based on the second temperature. Controlling the first switch based on the first temperature may include closing the first switch when the first temperature is above a first value and controlling the second switch based on the second temperature may include closing the second switch when the second temperature is above the first value. Controlling the first switch based on the first temperature may include opening the first switch when the first temperature is below the first value and controlling the second switch based on the second temperature may include opening the second switch when the second temperature is below the first value.

Applying electrical energy from the source of electrical energy to the first probe in a manner that causes the first probe to create a lesion in the tissue into which the first probe is inserted may include applying electrical energy to the first probe when the first temperature is below a second value and removing the applied electrical energy from the first probe when the first temperature is above the second value Applying electrical energy from the source of electrical energy to the second probe in a manner that causes the second probe to create a lesion in the tissue into which the second probe is inserted may include applying electrical energy to the second probe when the second temperature is below the second value and removing the applied electrical energy from the second probe when the second temperature is above the second value.

Applying electrical energy to the first probe may include closing a third switch, with the third switch being coupled between the first probe and the source of electrical energy such that the first probe is disconnected from the source of electrical energy when the third switch is in an open state and connected to the source of electrical energy when the third switch is in a closed state. Removing the applied electrical energy from the first probe may include opening the third switch. Applying electrical energy to the second probe may include closing a fourth switch, with the fourth switch being coupled between the second probe and the source of electrical energy such that the second probe is disconnected from the source of electrical energy when the fourth switch is in an open state and connected to the source of electrical energy when the fourth switch is in a closed state. Removing the applied electrical energy from the second probe may include opening the fourth switch.

The electrical energy applied to the first probe may be pulse width modulated by opening and closing the third switch. The electrical energy applied to the second probe may be pulse width modulated by opening and closing the fourth switch.

An amount of power applied to the first probe or the second probe may be controlled by controlling a magnitude of a voltage output by the source of electrical energy. Applying electrical energy to the first probe may include causing the source of electrical energy to output a voltage with a non-zero magnitude and removing the applied electrical energy from the first probe may include causing the source of electrical energy to output a voltage with a zero magnitude. Similarly, applying electrical energy to the second probe may include causing the source of electrical energy to output a voltage with a non-zero magnitude and removing the applied electrical energy from the second probe may include causing the source of electrical energy to output a voltage with a zero magnitude.

Applying electrical energy from the source of electrical energy to the first probe in a manner that causes the first probe to create a lesion in the tissue into which the first probe is inserted may include closing a third switch when the first parameter is below a first value and opening the third switch when the first parameter is above the first value. The third switch may be coupled between the first probe and the source of electrical energy such that the first probe is disconnected from the source of electrical energy when the third switch is in an open state and connected to the source of electrical energy when the third switch is in a closed state. Likewise, applying electrical energy from the source of electrical energy to the second probe in a manner that causes the second probe to create a lesion in the tissue into which the second probe is inserted may include closing a fourth switch when the second parameter is below the first value and opening the fourth switch when the second parameter is above the first value. The fourth switch may be coupled between the second probe and the source of electrical energy such that the second probe is disconnected from the source of electrical energy when the fourth switch is in an open state and connected to the source of electrical energy when the fourth switch is in a closed state.

In one aspect, an electrosurgical system includes a source of electrical energy, a first probe coupled to the source of electrical energy, and a second probe coupled to the source of electrical energy. A first switch is coupled to the first probe and couples the first probe to ground when in a closed state. A second switch is coupled to the second probe and couples the second probe to ground when in a closed state. A control system is configured to receive an indication of a first temperature at the first probe and control the state of the first switch based on the first temperature. The control system is also configured to receive an indication of a second temperature at the second probe and control the state of the second switch based on the second temperature.

The details of one or more implementations are set forth in the accompanying drawings and the description below. Other features, objects, and advantages will be apparent from the description and drawings.

### DESCRIPTION OF DRAWINGS

FIG. 1 is a schematic of an RF electrosurgical system.

FIG. 2 is a graph of the voltages applied to the probes of the RF electrosurgical system.

FIG. 3 is a graph of temperature versus time at the probes of the RF electrosurgical system.

FIG. 4 is a schematic showing an alternative RF generation system for the RF electrosurgical system.

FIG. 5 is a schematic showing another alternative RF generation system for the RF electrosurgical system.

FIG. 6 is an illustration depicting the use of RF lesioning to treat back pain caused by facet joint syndrome.

### DETAILED DESCRIPTION

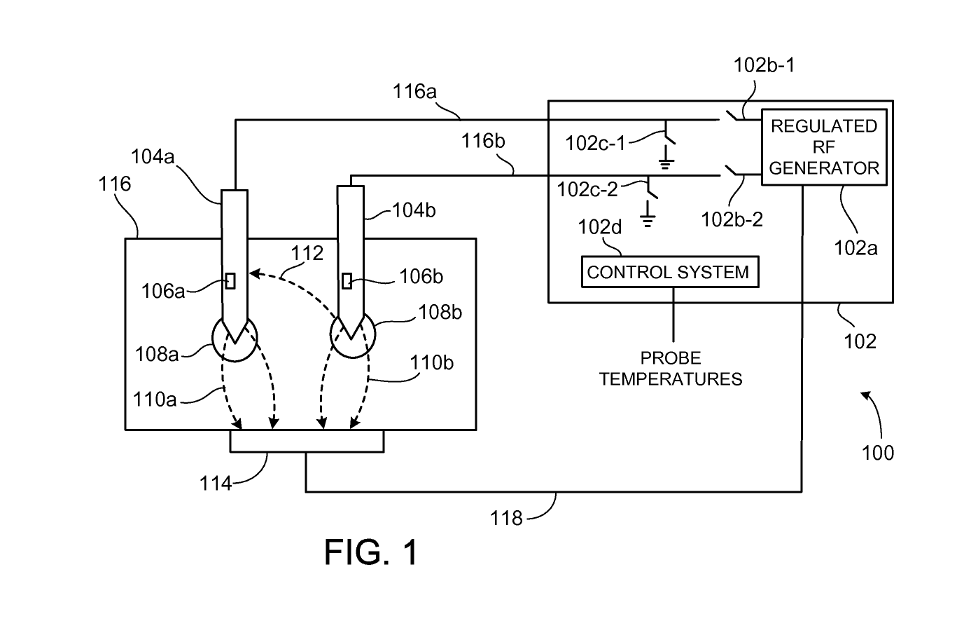

Referring to FIG. 1, an electrosurgical system 100, such as an RF lesioning system, includes an RF generation system 102, a first RF probe 104a, a second RF probe 104b, and a ground pad 114. Probes 104a and 104b include temperature sensors 106a and 106b (for example, T-type thermocouples), and are coupled to RF generation system 102 through leads 116a and 116b, respectively. The ground pad 114 is coupled to RF generation system 102 through a lead 118. The RF generation system 102 includes an RF generator 102a, which may be regulated to maintain a constant RF voltage waveform. The RF generation system 102 also includes source AC switches 102b-1 and 102b-2 and ground AC switches 102c-1 and 102c-2. As described more fully below, the ground AC switches 102c-1 and 102c-2 can provide an alternate path to ground for cross-talk currents, which can reduce or eliminate the effects of the cross-talk currents on the temperatures at the tips of the probes 104a and 104b.

The RF generation system 102d includes a control system 102d to control the state of the source AC switches 102b-1 and 102b-2 and the ground AC switches 102c-1 and 102c-2. The control system 102d may be implemented, for example, using a microprocessor or microcontroller. The control system 102d receives temperature readings from temperature sensors 106a and 106b. As described in more detail below, based on those temperature readings, the control system controls the operation of the source AC switches 102b-1 and 102b-2 and ground AC switches 102c-1 and 102c-2 to maintain the temperatures at the probes 104a and 104b at or near a target temperature.

To perform RF lesioning, the RF probes 104a and 104b are inserted into human tissue 116 and each probe is situated in or near the tissue to be lesioned. For example, if the procedure being performed is rhizotomy, the probes 104a and 104b are each positioned near a nerve to be lesioned (not shown) using, for example, fluoroscopy. The probes 104a and 104b may be placed, for example, within 5 mm of the nerve for a 10 mm diameter lesion size. More generally, the probes 104a and 104b are positioned so that the distance to the nerve is within the lesion size. The ground pad 114 is also attached to the patient's body.

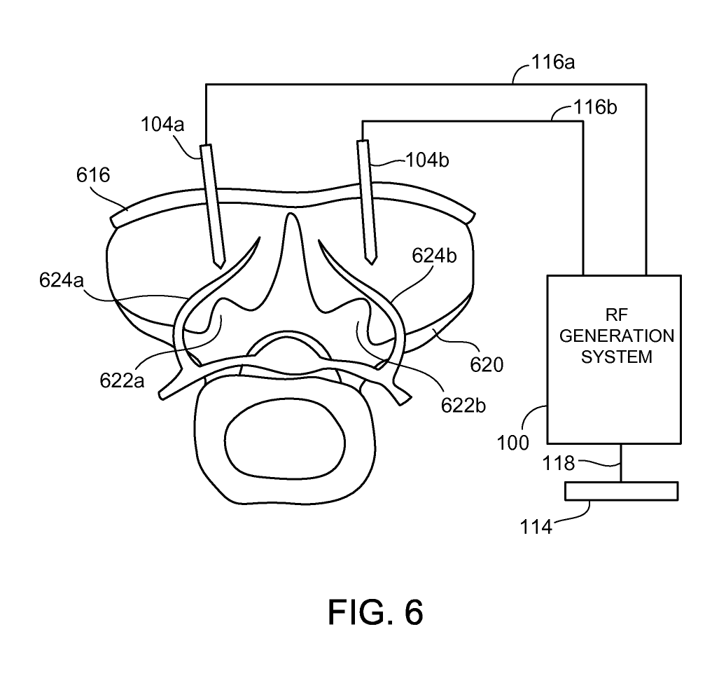

Depending on the procedure, the physician can first place the RF generation system in a diagnostic mode to insure proper placement of the probes 104a and 104b. For example, if the device 100 is used to lesion nerves, a diagnostic mode (described in more detail with respect to FIG. 6) can be used to insure that the probes 104a and 104b are placed near the proper nerves. Once the probes 104a and 104b are properly positioned, the physician places the RF generation system 102 into a RF Lesion mode. At this point, the control system 102d closes or maintains closed both source AC switches 102b-1 and 102b-2, and opens or maintains open the ground AC switches 102c-1 and 102c-2. The control system 102d then causes the regulated RF generator to apply, for example, a continuous RF voltage to each probe 104a and 104b through the closed source AC switches 102b-12 and 102b-2.

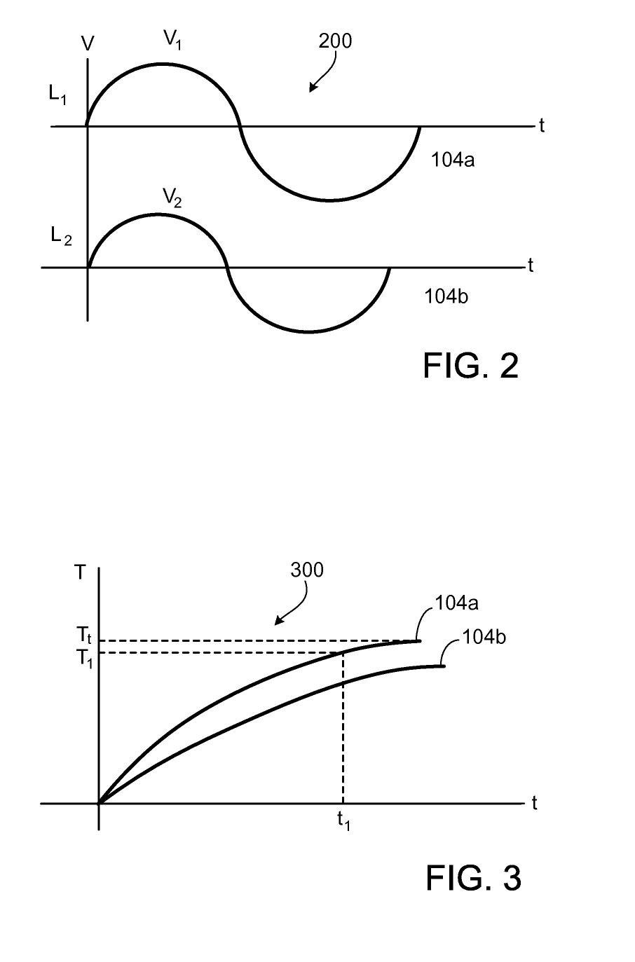

Referring to FIG. 2, as shown by the graph 200, the RF generator 102a applies the same RF voltage signal to each probe 104a and 104b. For instance, a continuous RF voltage with a frequency of 460 KHz and a peak voltage of 65 Vrms can be applied to the probes 104a and 104b. Other frequencies and voltages may equally be used. Because the same RF signal is applied to each probe, the voltages at the probes 104a and 104b are substantially phase synchronous. This results in the voltage at each probe 104a and 104b being substantially the same at any given moment.

Referring again to FIG. 1, the application of the RF voltage to the probes 104a and 104b results in current flow 110a and 110b from the tips of probes 104a and 104b, respectively, to ground pad 114. Because the voltages at each probe 104a and 104b are substantially the same, a substantially zero potential difference exists between the probes 104a and 104b and substantially all of the current flows from the probes 104a and 104b to the ground pad 114. The current flow is generally related to the impedance between the ground pad 114 and the probes 104a and 104b, which is typically on the order of about 200 to about 500 Ohms. When a voltage with a peak magnitude of about 65 Vrms is used, this can result in peak currents between 200 and 700 mA. The current flow 110a and 110b causes heating of the tissue near the tips of probes 104a and 104b, which forms lesions 108a and 108b, respectively.

To properly create the lesions 108a and 108b without collateral damage to surrounding tissue, the temperature at the tips of the probes 108a and 108b is raised to and maintained within a threshold amount of a particular target temperature for a certain duration. The target temperature is generally between about 75 degrees Celsius and about 90 degrees Celsius, and the duration between about 30 to about 120 seconds, although longer durations can be used. In a particular embodiment, the target temperature is 80 degrees Celsius and the duration is 120 seconds. The threshold amount is, for example, plus or minus two degrees Celsius.

The control system 102d receives temperature readings from the temperature sensors 106a and 106b and when the temperature at a probe 104a or 104b raises to within the threshold amount of the target temperature, the control system 102d opens the corresponding source AC switch 102b-1 or 102b-2 to cut-off the supply of RF energy to that probe.

Referring to FIG. 3, as shown by graph 300, the temperature T at one of the probes may reach the lower threshold T₁ around the target temperature T₁ faster than the other probe. In the example shown, the temperature at probe 104a reaches the lower threshold T₁ at time t₁, while the temperature at probe 104b at time t₁ is still below the lower threshold T₁. This difference can be caused, for example, by the differences in impedances between the probe 104a and the ground pad 114 and the probe 104b and the ground pad 114, which can result in a greater current flow through the probe with the least impedance between it and the ground pad.

Following the example illustrated in FIG. 3, when the temperature at probe 104a reaches the lower threshold T₁ at t₁, the control system 102d opens source AC switch 102b-1, while maintaining source AC switch 102b-2 closed and ground AC switches 102c-1 and 102c-2 open. Opening the source AC switch 102b-1 disconnects probe 104a from the RF generator 102a.

Referring again to FIG. 1, when the source AC switch 102b-1 is opened and no voltage is applied to the probe 104a, a potential difference exists between the probe 104a and the probe 104b. As a result of the potential difference, a cross-talk current 112 flows from the probe 104b to the probe 104a. With the ground AC switch 102c-1 open, the cross-talk current 112 flows through the probe 104a to the ground pad 114. In that case, the cross-talk current 112 causes the temperature at the tip of the probe 104a to continue increasing above the target temperature Tₜ, which, if uncorrected, can result in collateral tissue damage.

To reduce or eliminate the temperature increase at the probe 104a as a result of cross-talk currents, the control system 102d closes the ground AC switch 102c-1 when the temperature at the probe 104a exceeds the upper threshold amount. The system 100 is designed so that the impedance between the probe 104a through the ground AC switch 102c-1 is less than the impedance between the probe 104a and the ground pad 114. As a result, the cross-talk current 112 flows from the probe 104a through the switch 102c-1 into ground, instead of flowing from the probe 104a through the tissue 116 to the ground pad 114. This can reduce or eliminate the increase in temperature caused by cross-talk currents.

If the temperature at the probe 104a then decreases below the upper threshold amount, the ground AC switch 102c-1 is opened. If the temperature at the probe 104a continues to drop below the lower threshold amount, then the control system 102d closes the source AC switch 102b-1 to reconnect the RF source to the probe 104a. This results in an increase of the temperature at the probe 104a. Once the temperature at the probe 104a raises to within the lower threshold amount, the source AC switch 102b-1 is opened again. The control system 102d continues to control the source AC switch 102b-1 and the ground AC switch 102c-1 in the same fashion until the end of the procedure.

The control system 102d also controls the source AC switch 102b-2 and ground AC switch 102c-2 in the same fashion. In particular, when the temperature at the probe 104b is within the lower threshold amount, the control system 102d opens the source AC switch 102b-2 and keeps the ground AC switch 102c-2 opened until the temperature at the probe 104b exceeds the upper threshold, at which point the ground AC switch 102c-2 is closed. As a result, temperature increases due to cross-talk between the probes 104a and 104b can be controlled by providing an alternate path for that current, namely, from the probes 104a and 104b to ground through the ground AC switches 102c-1 and 102c-2, respectively, rather than through the tissue 116 to the ground pad 114.

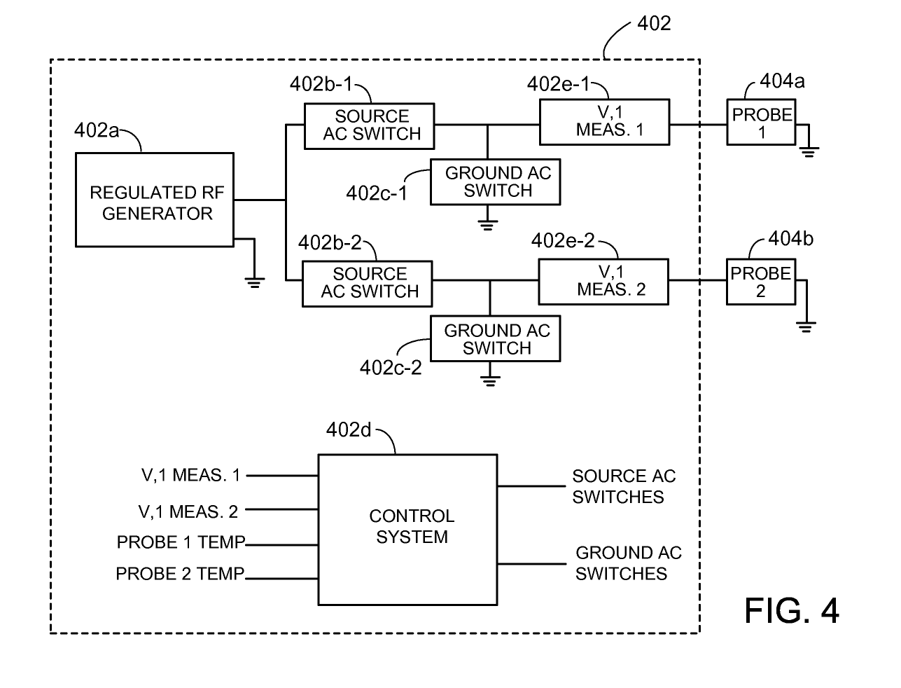

Referring to FIG. 4, in another embodiment, an RF generation system 402 also includes a voltage and current measurement network 402e-1 coupled to the probe 404a and a voltage and current measurement network 402e-2 coupled to the probe 404b. These networks 402e-1 and 402e-2 are used to the measure the voltage and current provided to a given one of the probes 404a and 404b. The control system 102d uses the temperature readings from the sensors on probes 404a and 404b, the voltage measurements, and the current measurements to control the operation of the source AC switches 402b-1 and 402b-2 so as to control the power delivered to a given probe 404a and 404b.

In particular, as with system 102, when the temperature of a probe needs to be increased, the control system 402d closes the associated source switch 402b-1 or 402b-2. However, rather than applying constant power to the probes 404a and 404b by maintaining the source AC switch closed, the amount of power applied to a given probe 404a or 404b is controlled by rapidly opening and closing the source AC switch 402b-1 or 402b-2, effectively pulse width modulating (PWM) the RF signal applied to the probes 404a and 404b. The control system 402d implements a controller, such as a proportional-integral-derivative (PID) controller, that controls the PWM of a given one of the source AC switches 402b-1 and 402b-2, so as to control the power delivered, based on the lower threshold amount, and the temperature, voltage, and current measurement for that probe.

To measure the voltage and current for a given probe, the other probe may be isolated by opening the associated source AC switch 402b-1 or 402b-2 so that the RF voltage from the generator 402a is applied only to one of the probes, and the current returning to the RF generator is only the current flowing through that probe. When the other probes are isolated, the voltage and current measurement networks 402e-1 or 402e-2 for the non-isolated probe can detect the voltage and current being applied to that probe (which can also be used to obtain the power applied to that probe). The control system 402d can cycle through the probes to detect the voltage and current a certain number of times per second, such as five times per second. The total duration for one cycle can be, as an example, from 5 to 10 milliseconds.

The measured voltage and current for a given probe can also be used to determine the impedance between that probe and the ground pad. An impedance drop below a certain amount (for example, about 100 Ohms) may indicate a problem with the procedure. The control system 402d monitors this impedance for each probe, and if the impedance drops below a certain level, shuts-down the system 402 as a safety precaution.

Once the temperature of a probe is within the lower and upper threshold amounts, the control system 402d controls the source AC switches 402b-1 and 402b-2 and the ground AC switches 402c-1 and 402c-2 in the same fashion as described with respect to system 100.

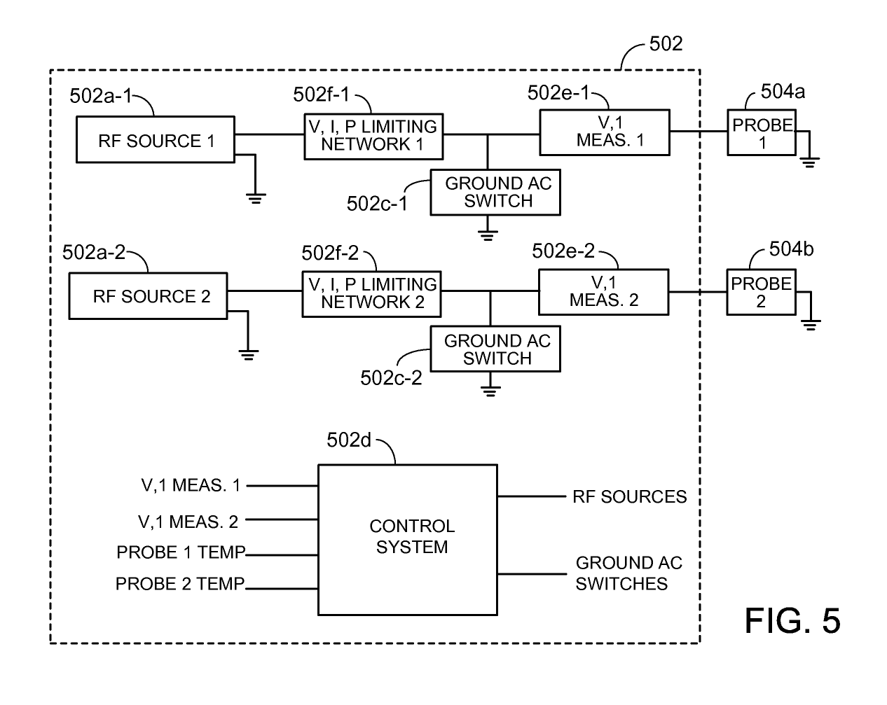

FIG. 5 is a schematic illustrating another embodiment of an RF generation system 502 in which the amount of power supplied to a probe is controlled through a controller. In system 502, independent RF sources 502a-1 and 502a-2 are used to provide RF voltages to probes 504a and 504b, respectively.

The independent RF sources 502a-1 and 502a-2 are unregulated RF sources and the magnitude of the RF voltages supplied by the sources 502a-1 and 502a-2 can be controlled by one or more control signals from the control system 502d. Because the RF sources 504a and 504b are unregulated, active or passive voltage, current, and power limiting networks 502f-1 and 502f-2 are included. These networks 502f-1 and 502f-2 limit the amount of voltage and current (and, hence, power) that can be transmitted through a given probe to help insure the safety of the patient.

System 502 includes a voltage and current measurement networks 502e-1 coupled to the probe 504a and a voltage and current measurement networks 402e-2 coupled to the probe 504b. Ground AC switches 502c-1 and 502c-2 are included in system 502, but source AC switches are not. To measure the voltage and current for a given probe, the other probe may be isolated by setting the magnitude of the voltage applied to the other probe to zero or switching off the corresponding RF source 502a-1 or 502a-2.

System 502 operates in a similar fashion as system 402. However, instead of controlling the amount of power supplied to a given probe by using source AC switches, the amount of power provided to a given probe is controlled by controlling the magnitude of the voltage supplied from the associated RF source 502a-1 or 502a-2. Similar to the system 402, the control system 502d implements a controller, such as a PID controller, that controls power supplied to a given probe. However, instead of controlling the PWM of a source AC switch, the controller changes the magnitude of the voltage supplied from the associated RF source based on the lower threshold amount, and the temperature, voltage, and current measurement for that probe.

Also, once the temperature of a probe is above the lower threshold amount, the control system 502d sets the magnitude of the associated RF source 502a-1 or 502a-2 to zero to cut off the supply of energy to that probe, rather than opening a source AC switch. The control system 502d controls the ground AC switches 502c-1 and 502c-2 in the same fashion as described with respect to systems 102 and 402.

FIG. 6 is an illustration depicting the use of the electrosurgical device 100 to treat back pain caused by facet joint syndrome. A given vertebra 620 of the spinal column includes a pair of joints 622a and 622b, referred to as facet joints. These joints connect a given level of the spinal column to the levels above and below that level. On a given level, one or both of the facet joints 622a and 622b can become inflamed due to injury and/or arthritis, resulting in potentially severe back pain.

To treat this pain, the probe 104a is inserted through the skin and muscle 616 of the back and placed near the medial branch nerve 624a that supplies the facet joint 622a. While not shown, the probe 104a may be inserted and placed near the medial branch nerve 624a using an introducer cannula. The physician may use fluoroscopy to aid in the placement of the cannula or probe 104a. The ground pad 114 may be placed on the patient's body. Typically, with facet joint syndrome, both of the facet joints of a given level are inflamed and causing pain. If this is the case, the second probe 104b is also inserted through the skin and muscle 616 and placed near the medial branch nerve 624b that supplies the other facet joint 622b. Using both probes 104a and 104b simultaneously to lesion both nerves 624a and 624b can reduce the amount of time taken to perform the procedure, which can be desirable because the lesioning process can be painful for the patient. Also, reduction of procedure time may provide significant cost advantages.

After the initial placement of the probes 104a and 104b, the physician places the RF generation system 102 in a diagnostic mode to insure proper placement of the probes 104a and 104b. In the diagnostic mode, a low level of RF energy is separately applied to each probe 104a and 104b to cause sensory stimulation and motor stimulation. For example, the physician can use the RF generation system 102 to separately apply a pulsed RF voltage to each probe 104a and 104b with a peak magnitude of 0-1 Vrms, a base frequency of 460 KHz, a pulse frequency of 50 Hz, and a pulse duration of 0.1-3 ms to perform sensory stimulation. After sensory stimulation is complete, the physician can use the RF generation system to separately apply a pulsed RF voltage to each probe 104a and 104b with a peak magnitude of 0-10V, a base frequency of 460 KHz, a pulse frequency of 2 Hz, and a pulse duration of 0.1-3 ms to perform motor stimulation.

If the results of the sensory and motor stimulations indicate to the physician that the probes 104a and 104b are properly positioned, the physician then places the RF generation system 102 in the destructive mode with the RF generation system 102 operating as described above to control the temperatures at the probes 104a and 104b to effect lesioning, while reducing the effects of cross-talk between the probes 104a and 104b. If either of the RF generation systems 402 or 502 is used, then the RF generation system 402 or 502 controls the RF power provided to the probes 104a and 104b, in addition to reducing the effects of cross-talk.

A number of implementations have been described. Nevertheless, it will be understood that various modifications may be made. For example, while control systems 102d, 402d, and 502d are described as being implemented with a microprocessor or microcontroller, these control systems can alternatively be implemented using analog circuitry or other digital circuitry, such as an FPGA or ASIC. Also, while the control systems 102d, 204d, and 502d are described as implementing a PID controller, other control schemes can be used, such as a proportional-integral (PI) controller.

Furthermore, the above described implementations control the power supplied to the probes and the ground AC switches based on the temperature at the probes. Other implementations can, alternatively or additionally, control the power and/or ground AC switch based on different parameters. For example, in one implementation, a source AC switch and a ground AC switch for each probe are controlled based on the current through that probe. Generally, as a lesion forms, the impedance in the probe decreases and the current increases.

This implementation includes an RF generation system configured similar to the system 400 of FIG. 4, except that temperature sensors are not included on the probes 404a and 404b or are included but not utilized. When the procedure starts in this implementation, the source AC switches are closed and the ground AC switches are opened. Power is applied to each probe, and the current through each probe is measured by opening the source AC switch for the other probe to isolate the probe to be measured, as described above with respect to the implementation of FIG. 4. When the current through a probe exceeds a threshold current (for example, a current in the range of 100-150 mA), the source AC switch for that probe is opened to stop the supply of current to that probe. Once the source AC switch is opened, cross-talk current may flow through the probe. If this cross-talk current exceeds the threshold current, the ground AC switch is closed to divert the cross-talk current to ground without passing through the tip of the probe.

In an alternative implementation, rather than using a source AC switch, the voltage of the RF source is controlled to keep the current below the threshold current when power is applied to the probe, and the ground AC switch is closed when the cross-talk current exceeds the threshold current. This implementation includes an RF generation system configured similar to the system 500 of FIG. 5. When the procedure starts, the ground AC switches are opened and the same voltage is applied to each probe. The current through each probe is measured by switching off the RF source or setting the magnitude of the voltage to zero for the other probe to isolate the probe to be measured. When the current through a probe exceeds a threshold current (for example, a current in the range of 100-150 mA), the magnitude of the voltage applied to the probe is reduced to maintain the current below the threshold current. If the magnitude is reduced to zero, but the current still exceeds the current threshold, then the ground AC switch is closed to divert any cross-talk current to ground without passing through the tip of the probe.

Other implementations may use, for example, the voltage or impedance at each probe to control the power and/or ground AC switches.

In addition, while two probes have been described, the methodology for reducing the effects of cross-talk can be extended to more than two probes. For example, often facet joint syndrome includes not only the inflammation of the facet joints of a given level of the spine, but also the inflammation of the facet joints above or below that level. In this situation, three, four, five, or six probes can be used as appropriate to treat the inflamed facet joints simultaneously, while ground AC switches are used to direct cross-talk current into ground without passing through the tissue to the ground pad.

Also, various features of the described embodiments of the RF generation systems can be used together. For instance, voltage and current limiting networks can be used with a regulated RF generator. Also, source AC switches can be used to control power delivery even if controllable, unregulated RF sources are used. While RF generation system 502 uses multiple unregulated RF sources, a single unregulated RF source can be used. Similarly, while RF generation systems 102 and 402 use a single regulated RF generator, multiple regulated RF generators can be used instead.

While the ground AC and source AC switches have been illustrated as being housed with the RF generator, any combination of these switches can be placed at other locations in the system. For instance, the ground AC switch for a probe can be included in a handle associated with the probe, rather than being housed in the RF generation system.

Furthermore, while specific procedures have been describe, the electrosurgical devices described above may be used for other procedures.

One or more, of the implementations may provide certain advantages. For example, one or more implementations may allow the RF energy to be applied to a probe more continuously than in other system designs; Providing a more continuous application of RF energy may be desirable because doing so may have a better therapeutic effect during certain procedures, such as denervation.

Some systems with multiple probes may be designed to multiplex the RF energy to each probe. In this case, RF energy is applied consecutively to each probe for a period of time, until the last probe is reached, at which point the cycle is started again with the first probe. In a system with four probes, for instance, the RF energy may be applied consecutively to each probe for about 1 millisecond, resulting in each probe receiving RF energy every 5 milliseconds. Once the temperature at a probe is at or near the target temperature, the probe is included, for example, only once every two to three cycles, so that RF energy is applied every 10-15 milliseconds to maintain the temperature near the target temperature.

Because multiplexed systems continuously cycle through applying RF energy to each probe, some or all of the implementations described above (or other implementations) may provide a more continuous application of RF energy than a multiplexed system. For instance, system 400 provides continuous RF energy until the temperature at the probe nears the target temperature, at which time the corresponding source AC switch is switched on and off to control the power delivered until the lower threshold is reach and the source AC switch is maintained open. Even though the application of RF energy is not continuous until the lower threshold is reached, the RF energy is applied more continuously than in a multiplexed system. As another example, system 500 provides continuous RF energy to each probe until the lower threshold of the target temperature is reached.

Other system designs may employ pulsed RF energy, in which the RF energy is periodically applied to each probe for a certain duration. For example, the RF energy may be applied to each probe for 1 millisecond every 1 second. The “on” pulses may be applied to each probe at the same time or at different times. Some or all of the implementations may provide a more continuous application of RF energy than pulsed RF systems.

A number of implementations have been described. Nevertheless, it will be understood that various modifications may be made. Accordingly, other implementations are within the scope of the following claims.

What is claimed is:

1. An electrosurgical system comprising:
a source of electrical energy;
a grounding pad coupled to the source of electrical energy and configured to be coupled to a body of a patient;
a first probe coupled to the source of electrical energy and configured to be inserted into tissue of the patient, wherein the first probe is further configured to create a lesion when the first probe is inserted into tissue and electrical energy is applied to the first probe from the source of electrical energy;
a second probe coupled to the source of electrical energy and configured to be inserted into tissue of the patient, wherein the second probe is further configured to create a lesion when the second probe is inserted into tissue and electrical energy is applied to the second probe from the source of electrical energy;
a first switch coupled to the first probe such that the first switch couples the first probe to ground when in a closed state;
a second switch coupled to the second probe such that the second switch couples the second probe to ground when in a closed state;
a control system configured to:
apply electrical energy from the source of electrical energy to the first probe in a manner that causes the first probe to create a lesion when the first probe is inserted into tissue;
apply electrical energy from the source of electrical energy to the second probe in a manner that causes the second probe to create a lesion when the second probe is inserted into tissue;
receive an indication of a first parameter associated with the first probe;
control the first switch based on the first parameter;
receive an indication of a second parameter associated with the second probe; and
control the second switch based on the second parameter.

2. The system of claim 1 wherein:
the first parameter includes a first temperature at the first probe such that the control system is configured to control the first switch based on the first temperature; and
the second parameter includes a second temperature at the second probe such that the control system is configured to control the second switch based on the second temperature.

3. The system of claim 2 wherein:
to control the first switch based on the first temperature, the control system is configured to close the first switch when the first temperature is above a first value; and
to control the second switch based on the second temperature, the control system is configured to close the second switch when the second temperature is above the first value.

4. The system of claim 3 wherein:
to control the first switch based on the first temperature, the control system is configured to open the first switch when the first temperature is below the first value; and
to control the second switch based on the second temperature, the control system is configured to open the second switch when the second temperature is below the first value.

5. The system of claim 4 wherein:
to apply electrical energy from the source of electrical energy to the first probe in a manner that causes the second probe to create a lesion when the first probe is inserted into tissue, the control system is configured to apply electrical energy to the first probe when the first temperature is below a second value and remove the applied electrical energy from the first probe when the first temperature is above the second value;
to apply electrical energy from the source of electrical energy to the second probe in a manner that causes the second probe to create a lesion when the second probe is inserted into tissue, the control system is configured to apply electrical energy to the second probe when the second temperature is below the second value and remove the applied electrical energy from the second probe when the second temperature is above the second value.

6. The system of claim 5 further comprising:
a third switch coupled between the first probe and the source of electrical energy such that the first probe is disconnected from the source of electrical energy when the third switch is in an open state and connected to the source of electrical energy when the third switch is in a closed state;
a fourth switch coupled between the second probe and the source of electrical energy such that the second probe is disconnected from the source of electrical energy when the fourth switch is in an open state and connected to the source of electrical energy when the fourth switch is in a closed state; and
wherein:
to apply electrical energy to the first probe, the control system is configured to close the third switch;
to remove the applied electrical energy from the first probe, the control system is configured to open the third switch;
to apply electrical energy to the second probe, the control system is configured to close the fourth switch; and
to remove the applied electrical energy from the second probe, the control system is configured to open the fourth switch.

7. The system of claim 6 wherein the control system is configured to:
pulse width modulate the electrical energy applied to the first probe by opening and closing the third switch; and
pulse width modulate the electrical energy applied to the second probe by opening and closing the fourth switch.

8. The system of claim 5 wherein:
to apply electrical energy to the first probe, the control system is configured to cause the source of electrical energy to output a voltage with a non-zero magnitude;
to remove the applied electrical energy from the first probe, the control system is configured to cause the source of electrical energy to output a voltage with a zero magnitude;
to apply electrical energy to the second probe, the control system is configured to cause the source of electrical energy to output a voltage with a non-zero magnitude; and
to remove the applied electrical energy from the second probe, the control system is configured to cause the source of electrical energy to output a voltage with a zero magnitude.

9. The system of claim 1 wherein:
the first parameter includes a first current through the first probe such that the control system is configured to control the first switch based on the first current; and
the second parameter includes a second current through the second probe such that the control system is configured to control the second switch based on the second current.

10. The system of claim 9 wherein:
to control the first switch based on the first current, the control system is configured to open the first switch when the first current is below a first value and close the first switch when the first current is above the first value; and
to control the second switch based on the second current, the control system is configured to open the second switch when the second current is below the first value and close the second switch when the second current is above the first value.

11. The system of claim 1 further comprising:
a third switch coupled between the first probe and the source of electrical energy such that the first probe is disconnected from the source of electrical energy when the third switch is in an open state and connected to the source of electrical energy when the third switch is in a closed state;
a fourth switch coupled between the second probe and the source of electrical energy such that the second probe is disconnected from the source of electrical energy when the fourth switch is in an open state and connected to the source of electrical energy when the fourth switch is in a closed state; and
wherein:
to apply electrical energy to the first probe, the control system is configured to close the third switch;
to remove the applied electrical energy from the first probe, the control system is configured to open the third switch;
to apply electrical energy to the second probe, the control system is configured to close the fourth switch; and
to remove the applied electrical energy from the second probe, the control system is configured to open the fourth switch.

12. The system of claim 11 wherein the control system is configured to:
close the third switch when the first parameter is below a first value;
open the third switch when the first parameter is above the first value;
close the fourth switch when the second parameter is below the first value; and
open the fourth switch when the second parameter is above the first value.

13. The system of claim 11 wherein the control system is configured to:
pulse width modulate the electrical energy applied to the first probe by opening and closing the third switch; and
pulse width modulate the electrical energy applied to the second probe by opening and closing the fourth switch.

14. The system of claim 1 wherein the control system is configured to control an amount of power applied to the first probe or the second probe by controlling a magnitude of a voltage output by the source of electrical energy.

15. The system of claim 1 wherein:
the first probe includes a first probe tip;
the second probe includes a second probe tip;
the first probe and first switch are configured such that current flows from the first probe to ground without passing through the first probe tip when the first switch is closed; and
the second probe and second switch are configured such that current flows from the second probe to ground without passing through the second probe tip when the first switch is closed.

16. The system of claim 1 wherein:
the first probe and first switch are configured such that an impedance between the first probe and ground is less than an impedance between the first probe and the grounding pad when the first probe is inserted in the tissue of the patient and the first switch is closed; and
the second probe and second switch are configured such that an impedance between the second probe and ground is less than an impedance between the second probe and the grounding pad when the second probe is inserted in the tissue of the patient and the second switch is closed.

17. A method of performing electrosurgery comprising:
coupling a grounding pad to a body of a patient, wherein the grounding pad is coupled to a source of electrical energy;
inserting a first probe into tissue of the patient, wherein the first probe is coupled to the source of electrical energy and configured to create a lesion when the first probe is inserted into tissue and electrical energy is applied to the first probe from the source of electrical energy;
inserting a second probe into tissue of the patient, wherein the second probe is coupled to the source of electrical energy and configured to create a lesion when the second probe is inserted into tissue and electrical energy is applied to the second probe from the source of electrical energy;
applying electrical energy from the source of electrical energy to the first probe in a manner that causes the first probe to create a lesion in the tissue into which the first probe is inserted;
applying electrical energy from the source of electrical energy to the second probe in a manner that causes the second probe to create a lesion in the tissue into which the second probe is inserted;
receiving an indication of a first parameter associated with the first probe;
controlling a first switch based on the first parameter, wherein the first switch is coupled to the first probe such that the first switch couples the first probe to ground when in a closed state;
receiving an indication of a second parameter associated with the second probe; and
controlling a second switch based on the second parameter, wherein the second switch is coupled to the second probe such that the second switch couples the second probe to ground when in a closed state;

18. The method of claim 17 wherein:
the first parameter includes a first temperature at the first probe such that controlling the first switch comprises controlling the first switch based on the first temperature; and
the second parameter includes a second temperature at the second probe such that controlling the second switch comprises controlling the second switch based on the second temperature.

19. The method of claim 18 wherein:
controlling the first switch based on the first temperature comprises closing the first switch when the first temperature is above a first value; and
controlling the second switch based on the second temperature comprises closing the second switch when the second temperature is above the first value.

20. The method of claim 19 wherein:
controlling the first switch based on the first temperature comprises opening the first switch when the first temperature is below the first value; and
controlling the second switch based on the second temperature comprises opening the second switch when the second temperature is below the first value.

21. The method of claim 20 wherein:
applying electrical energy from the source of electrical energy to the first probe in a manner that causes the first probe to create a lesion in the tissue into which the first probe is inserted comprises applying electrical energy to the first probe when the first temperature is below a second value and removing the applied electrical energy from the first probe when the first temperature is above the second value;
applying electrical energy from the source of electrical energy to the second probe in a manner that causes the second probe to create a lesion in the tissue into which the second probe is inserted comprises applying electrical energy to the second probe when the second temperature is below the second value and removing the applied electrical energy from the second probe when the second temperature is above the second value.

22. The method of claim 21 wherein:
applying electrical energy to the first probe comprises closing a third switch, the third switch being coupled between the first probe and the source of electrical energy such that the first probe is disconnected from the source of electrical energy when the third switch is in an open state and connected to the source of electrical energy when the third switch is in a closed state;
removing the applied electrical energy from the first probe comprises opening the third switch;
applying electrical energy to the second probe comprises closing a fourth switch, the fourth switch being coupled between the second probe and the source of electrical energy such that the second probe is disconnected from the source of electrical energy when the fourth switch is in an open state and connected to the source of electrical energy when the fourth switch is in a closed state; and
removing the applied electrical energy from the second probe comprises opening the fourth switch.

23. The method of claim 22 further comprising:
pulse width modulating the electrical energy applied to the first probe by opening and closing the third switch; and
pulse width modulating the electrical energy applied to the second probe by opening and closing the fourth switch.

24. The method of claim 21 wherein:
applying electrical energy to the first probe comprises causing the source of electrical energy to output a voltage with a non-zero magnitude;
removing the applied electrical energy from the first probe comprises causing the source of electrical energy to output a voltage with a zero magnitude;
applying electrical energy to the second probe comprises causing the source of electrical energy to output a voltage with a non-zero magnitude; and
removing the applied electrical energy from the second probe comprises causing the source of electrical energy to output a voltage with a zero magnitude.

25. The method of claim 17 further comprising:
applying electrical energy from the source of electrical energy, to the first probe in a manner that causes the first probe to create a lesion in the tissue into which the first probe is inserted comprises closing a third switch when the first parameter is below a first value and opening the third switch when the first parameter is above the first value, the third switch being coupled between the first probe and the source of electrical energy such that the first probe is disconnected from the source of electrical energy when the third switch is in an open state and connected to the source of electrical energy when the third switch is in a closed state; and
applying electrical energy from the source of electrical energy to the second probe in a manner that causes the second probe to create a lesion in the tissue into which the second probe is inserted comprises closing a fourth switch when the second parameter is below the first value and opening the fourth switch when the second parameter is above the first value, the fourth switch being coupled between the second probe and the source of electrical energy such that the second probe is disconnected from the source of electrical energy when the fourth switch is in an open state and connected to the source of electrical energy when the fourth switch is in a closed state.

26. The method of claim 25 further comprising:
pulse width modulating the electrical energy applied to the first probe by opening and closing the third switch; and
pulse width modulating the electrical energy applied to the second probe by opening and closing the fourth switch.

27. The method of claim 17 further comprising controlling an amount of power applied to the first probe or the second probe by controlling a magnitude of a voltage output by the source of electrical energy.
